(19) United States
(12) Patent Application Publication
(10) Pub. No.: US 2018/0140174 A1
(43) Pub. Date: May 24, 2018

(54) BLOOD-VESSEL RECOGNITION SYSTEM

(71) Applicant: OLYMPUS CORPORATION, Tokyo (JP)

(72) Inventors: Takeshi WATANABE, Tokyo (JP); Mitsuru NAMIKI, Saitama (JP); Kenichi KIMURA, Kanagawa (JP)

(73) Assignee: OLYMPUS CORPORATION, Tokyo (JP)

(21) Appl. No.: 15/860,740

(22) Filed: Jan. 3, 2018

Related U.S. Application Data
(63) Continuation of application No. PCT/JP2016/070592, filed on Jul. 12, 2016.

(30) Foreign Application Priority Data
Jul. 14, 2015 (JP) 2015-140679

(51) Int. Cl. A61B 1/06; A61B 1/00; A61B 1/07; A61B 5/00; A61B 1/04

(52) U.S. Cl. CPC A61B 1/063; A61B 1/00006; A61B 1/07; A61B 1/0669; A61B 1/04; A61B 1/00045; A61B 5/489

(57) ABSTRACT

Unnecessary emission of strong laser light is automatically prevented. Provided is a blood-vessel recognition system including: a laser-light source; a blood-vessel recognition probe that has a probe body that is inserted into a body and a laser-light emitting unit that is provided on the probe body and that emits laser light supplied from the laser-light source; an in-use-state determining unit that determines whether the blood-vessel recognition probe is in an in-use state in which the laser light emitted from the laser-light emitting unit is radiated onto living tissue in the body; and a control unit that controls the laser-light source on the basis of a determination result obtained by the in-use-state determining unit, such that the intensity of the laser light is reduced when the blood-vessel recognition probe is not in the in-use state, compared with when the blood-vessel recognition probe is in the in-use state.

### CROSS-REFERENCE TO RELATED APPLICATIONS

This is a continuation of International Application PCT/JP2016/070592, with an international filing date of Jul. 12, 2016, which is hereby incorporated by reference herein in its entirety. This application claims the benefit of Japanese Patent Application No. 2015-140679, filed on Jul. 14, 2015, the content of which is incorporated herein by reference.

### TECHNICAL FIELD

The present invention relates to a blood-vessel recognition system.

### Background Art

In surgical treatment of living tissue, it is important for a surgeon to accurately recognize the existence of a blood vessel hidden in the inside of the living tissue and to perform treatment so as to avoid the blood vessel. Thus, surgical treatment systems having a function for optically detecting a blood vessel existing in living tissue have been proposed (for example, see PTL 1). In PTL 1, laser light for measurement is radiated onto living tissue, and the presence or absence of a blood vessel is detected on the basis of reflected light, scattered light, or fluorescence from the living tissue.

### CITATION LIST

### Patent Literature

{PTL 1} Publication of Japanese Patent No. 4490807

### SUMMARY OF INVENTION

### Technical Problem

An object of the present invention is to provide a blood-vessel recognition system capable of automatically preventing unnecessary emission of strong laser light.

### Solution to Problem

An aspect of the present invention provides a blood-vessel recognition system including: a laser-light source; a blood-vessel recognition probe that has a probe body that is inserted into a body and a laser-light emitting unit that is provided on the probe body and that emits laser light supplied from the laser-light source; an in-use-state determining unit that determines whether the blood-vessel recognition probe is in an in-use state in which the laser light emitted from the laser-light emitting unit is radiated onto living tissue in the body; and a control unit that controls the laser-light source on the basis of a determination result obtained by the in-use-state determining unit, such that the intensity of the laser light is reduced when the blood-vessel recognition probe is not in the in-use state, compared with when the blood-vessel recognition probe is in the in-use state.

The above-described aspect may further include an endoscope that is inserted into the body, wherein the in-use-state determining unit may determine that the blood-vessel recognition probe is in the in-use state when an image of the blood-vessel recognition probe is included in an endoscopic image acquired by the endoscope.

The above-described aspect may further include: a detection-light source that outputs detection light having a lower intensity than the intensity of the laser light, which is output from the laser-light source when the blood-vessel recognition probe is in the in-use state; and a detection-light detecting unit that detects the detection light radiated onto the living tissue, wherein the blood-vessel recognition probe may be provided with a detection-light emitting unit that is provided on the probe body and that radiates, onto the living tissue, the detection light supplied from the detection-light source; and the in-use-state determining unit may determine that the blood-vessel recognition probe is in the in-use state when the detection-light detecting unit detects the detection light.

The above-described aspect may further include: an endoscope that is inserted into the body; a detection-light source that outputs detection light having a lower intensity than the intensity of the laser light, which is output from the laser-light source when the blood-vessel recognition probe is in the in-use state; and a detection-light detecting unit that is provided on the probe body and that detects the detection light radiated onto the living tissue, wherein the endoscope may be provided with a detection-light emitting unit that radiates, onto the living tissue, the detection light supplied from the detection-light source; and the in-use-state determining unit may determine that the blood-vessel recognition probe is in the in-use state when the detection-light detecting unit detects the detection light.

In the above-described aspect, the detection-light source may output the detection light, which has a property different from that of the laser light.

In the above-described aspect, the detection-light source may output the detection light whose intensity temporally changes; and the in-use-state determining unit may determine whether the blood-vessel recognition probe is in the in-use state on the basis of the temporal change in the intensity of the detection light detected by the detection-light detecting unit.

In the above-described aspect, the detection-light source may output the detection light, which has an intensity distribution having a predetermined pattern in the cross section intersecting the optical axis; the detection-light detecting unit may acquire an image of the living tissue; and the in-use-state determining unit may determine that the blood-vessel recognition probe is in the in-use state when an image of the predetermined pattern of the detection light is included in the image acquired by the detection-light detecting unit.

In the above-described aspect, the laser-light source may be capable of changing the intensity of the laser light between a first intensity and a second intensity that is lower than the first intensity; and the detection-light source may be formed of the laser-light source.

The above-described aspect may further include an orientation detecting unit that is provided on the probe body and that detects an orientation of the probe body, wherein the in-use-state determining unit may determine whether the blood-vessel recognition probe is in the in-use state on the basis of the orientation of the probe body detected by the orientation detecting unit.

The above-described aspect may further include a display unit that displays an in-use-state indication indicating a determination result obtained by the in-use-state determining unit.

### BRIEF DESCRIPTION OF DRAWINGS

FIG. 1 is a view showing the overall configuration of a blood-vessel recognition system according to a first embodiment of the present invention.

FIG. 2 is a flowchart showing control of a laser-light source in the blood-vessel recognition system shown in FIG. 1.

FIG. 3 is a view showing the overall configuration of a blood-vessel recognition system according to a second embodiment of the present invention.

FIG. 4 is a view showing the overall configuration of a modification of the blood-vessel recognition system shown in FIG. 3.

FIG. 5 is a view showing the overall configuration of another modification of the blood-vessel recognition system shown in FIG. 3.

FIG. 6 is a view showing the overall configuration of still another modification of the blood-vessel recognition system shown in FIG. 3.

FIG. 7 is a view showing the overall configuration of a blood-vessel recognition system according to a third embodiment of the present invention.

FIG. 8 is a view showing the overall configuration of a modification of the blood-vessel recognition system shown in FIG. 1.

### DESCRIPTION OF EMBODIMENTS

### First Embodiment

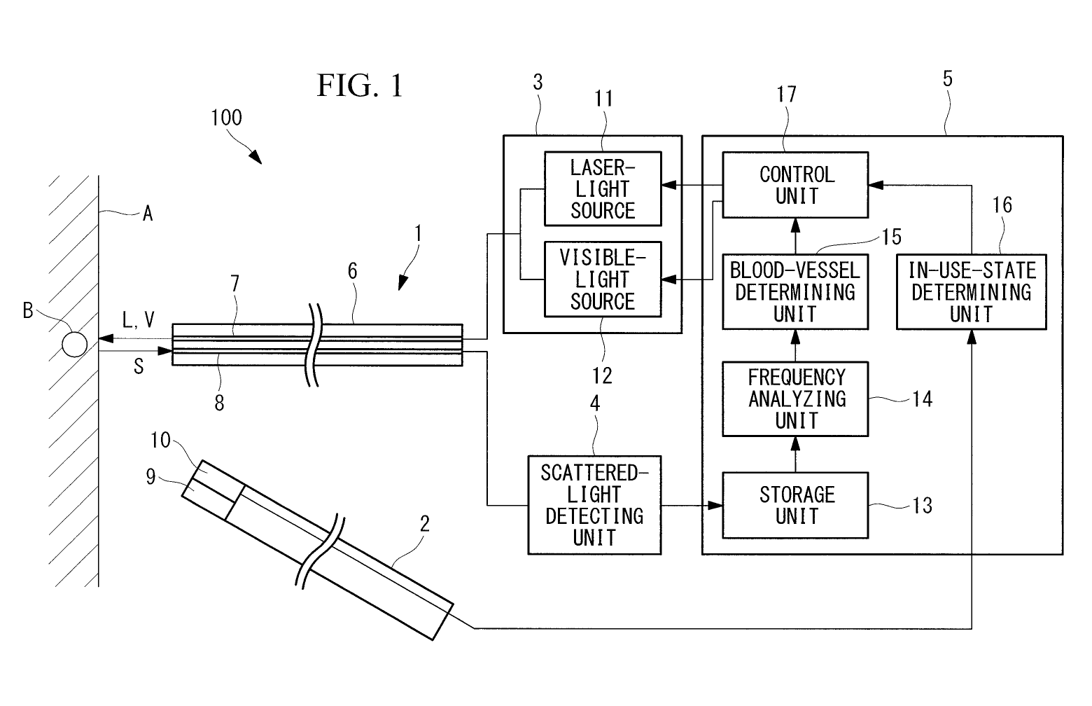

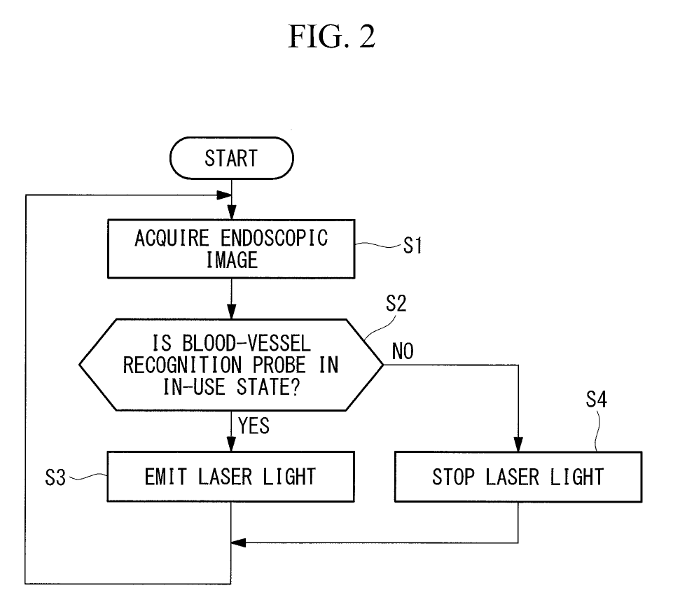

A blood-vessel recognition system 100 according to a first embodiment of the present invention will be described below with reference to FIGS. 1 and 2.

As shown in FIG. 1, the blood-vessel recognition system 100 of this embodiment is provided with: a blood-vessel recognition probe 1 and an endoscope 2 that are used by being inserted together into the body; a light source unit 3 that supplies laser light L and visible light V to the blood-vessel recognition probe 1; a scattered-light detecting unit 4 that detects scattered light S of the laser light L; and a control device 5 that controls the light source unit 3.

The blood-vessel recognition probe 1 is provided with an elongated probe body 6 that can be inserted into the body, and an irradiation optical fiber (laser-light emitting unit) 7 and a light-receiving optical fiber 8 that are provided along the longitudinal direction of the probe body 6.

The blood-vessel recognition probe 1 may be a treatment device, such as a high-frequency knife, for treating living tissue A. In this case, an action portion (not shown) for treating the living tissue A is provided at a distal end of the probe body 6.

A distal end of the irradiation optical fiber 7 is disposed in the vicinity of the distal end of the probe body 6, and a base end of the irradiation optical fiber 7 is connected to the light source unit 3. Laser light L and visible light V supplied from the light source unit 3 to the base end of the irradiation optical fiber 7 are emitted forward in the longitudinal direction of the probe body 6, from the distal end of the irradiation optical fiber 7.

A distal end of the light-receiving optical fiber 8 is disposed in the vicinity of the distal end of the probe body 6, so that scattered light S of the laser light L scattered by the living tissue A is received by the light-receiving optical fiber 8.

The endoscope 2 is provided with: an illumination unit 9 that emits illumination light from a distal end of the endoscope 2; and an image acquisition unit 10 that acquires an image of the living tissue A. Although FIG. 1 shows the flexible endoscope 2 of the videoscope-type, which is provided with the image acquisition unit 10 at the distal end thereof, instead of this, it is also possible to use a videoscope-type rigid endoscope that is provided with the image acquisition unit 10 at a base end thereof and in which reflected light of illumination light from the living tissue A is relayed to the image acquisition unit 10 by a relay optical system. Alternatively, it is also possible to use a fiberscope-type endoscope that is provided with the image acquisition unit 10 at a base end thereof and in which reflected light of illumination light from the living tissue A is transferred to the image acquisition unit 10 by means of an optical fiber.

The image acquisition unit 10 is provided with, for example, an image-acquisition optical system (not shown) that forms an image of reflected light of illumination light from the living tissue A, and an image-acquisition element (not shown) that captures the image of the living tissue A formed by the image-acquisition optical system to acquire an endoscopic image. The endoscopic image acquired by the image acquisition unit 10 is sent to an in-use-state determining unit 16 (to be described later) in the control device 5.

The light source unit 3 is provided with a laser-light source 11 that outputs laser light L, a visible-light source 12 that outputs visible light V having a wavelength in a visible region, and an optical multiplexer (not shown) that multiplexes the laser light L and the visible light V to make them enter the irradiation optical fiber 7.

The laser-light source 11 outputs laser light L in a wavelength region (for example, near-infrared region) that is less absorbed by blood.

It is preferred that the visible-light source 12 be a laser-light source. It is preferred that the color of visible light V be a color with which a surgeon can easily recognize the visible light V radiated onto the living tissue A, for example, green or blue.

The scattered-light detecting unit 4 is provided with a photodetector, such as a photodiode or a photomultiplier tube. The scattered-light detecting unit 4 is connected to a base end of the light-receiving optical fiber 8 and converts the intensity of scattered light S received by the light-receiving optical fiber 8 into a digital value. The obtained digital value is sent to a storage unit 13 (to be described later) in the control device 5.

The control device 5 is provided with: the storage unit 13, which accumulates data about the intensity of the scattered light S detected by the scattered-light detecting unit 4; a frequency analyzing unit 14 that performs frequency analysis for the data accumulated in the storage unit 13; a blood-vessel determining unit 15 that determines the presence or absence of a blood vessel B on the basis of the frequency analysis result obtained by the frequency analyzing unit 14; the in-use-state determining unit 16, which determines whether or not the blood-vessel recognition probe 1 is in the in-use state on the basis of an endoscopic image; and a control unit 17 that controls the laser-light source 11 and the visible-light source 12.

The control device 5 is a computer, for example, and is provided with a central processing unit (CPU), a main storage device, such as a RAM, and an auxiliary storage device. The auxiliary storage device is a non-transitory storage medium, such as a hard disk drive, and stores a program for causing the CPU to execute processing, to be described later, of the frequency analyzing unit 14, the blood-vessel determining unit 15, the in-use-state determining unit 16, and the control unit 17. When this program is loaded from the auxiliary storage device to the main storage device and is activated, the CPU executes the processing of the respective units 14, 15, 16, and 17 according to the program. Alternatively, the processing of the respective units 14, 15, 16, and 17 may be realized by an FPGA (field programmable gate array) or a PLD (programmable logic device) or may be realized by dedicated hardware, such as an ASIC (application specific integrated circuit).

The storage unit 13 stores, in time series, digital values received from the scattered-light detecting unit 4, thereby generating time-series data that indicates the temporal change in the intensity of the scattered light S.

The frequency analyzing unit 14 periodically obtains the time-series data from the storage unit 13 and subjects the obtained time-series data to a fast Fourier transform, thus obtaining a frequency spectrum. The frequency analyzing unit 14 obtains a frequency spectrum function F(ω) that indicates the relationship between the frequency ω and the intensity, and calculates the average frequency of the frequency spectrum F(ω) on the basis of the following expression (1). The calculated average frequency is sent to the blood-vessel determining unit 15.

$$\begin{matrix}\{ Expression 1 \} & \\ Average frequency = \frac{\int ω F ( ω ) d ω}{\int F ( ω ) d ω} & ( 1 )\end{matrix}$$

Here, the time-series data and the frequency spectrum will be described.

The living tissue A includes static components that are static, such as fat and leaking blood leaked from the blood vessel B through bleeding, and dynamic components that are moving, such as red blood cells in blood that flows in the blood vessel B. When the laser light L having a frequency f is radiated onto the static components, scattered light S having the same frequency f as the laser light L is produced. On the contrary, when the laser light L having the frequency f is radiated onto the dynamic components, scattered light S having a frequency f+Δf that is shifted from the frequency f of the laser light L due to the Doppler shift is produced. The frequency shift Δf at this time depends on the moving velocity of the dynamic components.

Therefore, when the blood vessel B is included in an area irradiated with the laser light L in the living tissue A, the light-receiving optical fiber 8 simultaneously receives scattered light S that is scattered by the blood in the blood vessel B, thus having the frequency f+Δf, and scattered light S that is scattered by the static components other than the blood in the blood vessel B, thus having the frequency f. As a result, time-series data shows beats in which the intensity of the scattered light S changes with a period corresponding to the shift Δf, due to the interference of the scattered light S having the frequency f and the scattered light S having the frequency f+Δf. Therefore, the time-series data is subjected to a fast Fourier transform, thereby obtaining, as a frequency spectrum, a Doppler spectrum having an intensity at the frequency corresponding to the velocity of the blood flow.

When there is no blood vessel B in an area irradiated with the laser light L, because the above-described beats do not occur, the Doppler spectrum becomes flat with no intensity in the entire frequency region. When there is a blood vessel B in which the blood flow is slow, the Doppler spectrum has an intensity in a low-frequency region. When there is a blood vessel B in which the blood flow is fast, the Doppler spectrum has an intensity in a high-frequency region. In this way, as the blood flow becomes faster, the average frequency of the Doppler spectrum becomes larger. It is known that the velocity of the blood flow in the blood vessel B is substantially proportional to the diameter of the blood vessel B. Therefore, the diameter of the blood vessel B can be estimated from the average frequency of the Doppler spectrum.

The blood-vessel determining unit 15 compares the average frequency received from the frequency analyzing unit 14 with a threshold. The threshold is an average frequency corresponding to the minimum value of the diameter of the detection-target blood vessel B. If the average frequency is equal to or greater than the threshold, the blood-vessel determining unit 15 determines that the blood vessel B exists and sends a TRUE signal to the control unit 17. On the other hand, if the average frequency is less than the threshold, the blood-vessel determining unit 15 determines that the blood vessel B does not exist and sends a FALSE signal to the control unit 17.

The in-use-state determining unit 16 determines whether the blood-vessel recognition probe 1 is in the in-use state on the basis of whether an image of the blood-vessel recognition probe 1 is included in an endoscopic image received from the image acquisition unit 10.

Specifically, the in-use-state determining unit 16 calculates a correlation value between the endoscopic image and each reference image stored in an image library (saving unit) (not shown) in which reference images obtained by capturing a distal end portion of the blood-vessel recognition probe 1 at various angles and distances are registered.

If the correlation value between the endoscopic image and at least one reference image is equal to or greater than a predetermined threshold, the in-use-state determining unit 16 determines that the blood-vessel recognition probe 1 is in the in-use state and sends a TRUE signal to the control unit 17. On the other hand, if the correlation values between the endoscopic image and all reference images are less than the predetermined threshold, the in-use-state determining unit 16 determines that the blood-vessel recognition probe 1 is not in the in-use state and sends a FALSE signal to the control unit 17.

The control unit 17 controls the visible-light source 12 on the basis of the determination result obtained by the blood-vessel determining unit 15 and controls the laser-light source 11 on the basis of the determination result obtained by the in-use-state determining unit 16.

Specifically, the control unit 17 causes the visible-light source 12 to output visible light V when the TRUE signal is received from the blood-vessel determining unit 15. On the other hand, the control unit 17 causes the visible-light source 12 to stop outputting the visible light V when the FALSE signal is received from the blood-vessel determining unit 15. Accordingly, only when the blood vessel B exists in an area irradiated with the laser light L, visible light V is emitted from the distal end of the irradiation optical fiber 7.

The control unit 17 causes the laser-light source 11 to output laser light L when the TRUE signal is received from the in-use-state determining unit 16. On the other hand, the control unit 17 causes the laser-light source 11 to stop outputting the laser light L when the FALSE signal is received from the in-use-state determining unit 16. Accordingly, only when it is determined that the blood-vessel recognition probe 1 is in the in-use state, laser light L is emitted from the distal end of the irradiation optical fiber 7.

Next, the operation of the thus-configured blood-vessel recognition system 100 will be described.

The blood-vessel recognition system 100 of this embodiment is used to recognize the distribution of the blood vessel B in a treatment area, before the living tissue A is treated by using the treatment device.

First, the endoscope 2 is inserted into the body via a trocar that is inserted into the body in advance, and the endoscope 2 is positioned such that the treatment area is included in an endoscopic image. Next, the blood-vessel recognition probe 1 is inserted into the body via another trocar. Accordingly, the distal end of the blood-vessel recognition probe 1 is included in the endoscopic image.

Next, the blood-vessel recognition probe 1 is moved so as to scan the laser light L emitted from the irradiation optical fiber 7 on the treatment area of the living tissue A. Scattered light S of the laser light L scattered by the living tissue A is received by the light-receiving optical fiber 8. The received scattered light S is detected by the scattered-light detecting unit 4, and time-series data of the scattered light S is generated in the storage unit 13. Next, in the frequency analyzing unit 14, a Doppler spectrum is obtained from the time-series data, and the average frequency of the Doppler spectrum is calculated. Next, the blood-vessel determining unit 15 determines whether the blood vessel B exists in an area irradiated with the laser light L in the living tissue A, on the basis of the average frequency.

When the blood-vessel determining unit 15 determines that the blood vessel B does not exist in the area irradiated with the laser light L, the control unit 17 causes only laser light L to be emitted from the irradiation optical fiber 7. When the blood-vessel determining unit 15 determines that the blood vessel B exists in the area irradiated with the laser light L, the control unit 17 causes visible light V, together with the laser light L, to be emitted from the irradiation optical fiber 7. Therefore, the surgeon can recognize the area irradiated with the visible light V, as an area where the blood vessel B exists.

Here, as shown in FIG. 2, the in-use-state determining unit 16 determines whether the blood-vessel recognition probe 1 is in the in-use state (Step S2) depending on whether an image of the blood-vessel recognition probe 1 is included in an endoscopic image (Step S1). Then, only when the blood-vessel recognition probe 1 is in the in-use state (YES in Step S2), laser light L is emitted (Step S3). When the blood-vessel recognition probe 1 is not in the in-use state (No in Step S2), emission of laser light L is stopped (Step S4).

Before the blood-vessel recognition probe 1 is inserted into the body, because the blood-vessel recognition probe 1 is not observed by the endoscope 2, emission of laser light L is stopped. When the blood-vessel recognition probe 1 is inserted into the body, an image of the blood-vessel recognition probe 1 appears in an endoscopic image, and emission of laser light L is started. Then, when the recognition operation for the blood vessel B using the blood-vessel recognition probe 1 is finished, and the blood-vessel recognition probe 1 is removed from the inside of the body, the image of the blood-vessel recognition probe 1 disappears from the endoscopic image, and the emission of the laser light L is stopped.

In this way, only when the blood-vessel recognition probe 1 is in the in-use state, in which recognition of the blood vessel B is performed by radiating laser light L onto the living tissue A in the body, the blood-vessel recognition probe 1 is observed by the endoscope 2. Therefore, it is possible to accurately determine whether the blood-vessel recognition probe 1 is in the in-use state on the basis of the endoscopic image. Accordingly, when the laser light L is not needed, e.g., when the blood-vessel recognition probe 1 is disposed outside the body, there is an advantage in that emission of the laser light L is automatically stopped, thus making it possible to automatically prevent the strong laser light L from being unnecessarily emitted.

When the blood-vessel recognition probe 1 is not in the in-use state, the control unit 17 may reduce the intensity of the laser light L and continue to cause the weak laser light L to be emitted, instead of stopping emission of the laser light L.

In this embodiment, although the in-use-state determining unit 16 determines the presence or absence of an image of the blood-vessel recognition probe 1 in an endoscopic image through image recognition based on reference images, instead of this, it is also possible to determine the presence or absence of an image of the blood-vessel recognition probe 1 by using another method.

Since the probe body 6 is coated with a sheath that is made of metal or the like and that has a high reflectance, an image of the blood-vessel recognition probe 1 has a high luminance value, compared with an image of the living tissue A, in an endoscopic image. Thus, the luminance value of an endoscopic image that includes the image of the blood-vessel recognition probe 1 becomes higher than the luminance value of an endoscopic image that does not include the image of the blood-vessel recognition probe 1. Therefore, the in-use-state determining unit 16 can determine whether the image of the blood-vessel recognition probe 1 is included in an endoscopic image, on the basis of the luminance value of the endoscopic image or the temporal change in the luminance value.

On the contour of the image of the blood-vessel recognition probe 1, the gradient of the luminance value becomes larger. Therefore, the in-use-state determining unit 16 may detect an edge from an endoscopic image and may determine that the image of the blood-vessel recognition probe 1 is included in the endoscopic image if the gradient of the luminance value at the detected edge is equal to or larger than a predetermined value.

Alternatively, an identification mark (for example, stripe lines at regular intervals) having a spatial periodic structure may be provided on the distal end portion of the blood-vessel recognition probe 1, and the in-use-state determining unit 16 may determine the presence or absence of an image of the blood-vessel recognition probe 1 on the basis of a spatial frequency included in an endoscopic image. The periodic structure of the identification mark has a spatial frequency different from a spatial frequency of the living tissue A. The in-use-state determining unit 16 subjects the endoscopic image to a fast Fourier transform and obtains a spatial frequency included in the endoscopic image, thereby making it possible to determine whether the identification mark is included in the endoscopic image.

In this embodiment, although the reference images obtained by capturing the distal end portion of the blood-vessel recognition probe 1 at various angles and distances are registered in the image library, instead of this, it is also possible to provide, in the vicinity of the distal end portion of the blood-vessel recognition probe 1, a logo mark for identifying the blood-vessel recognition probe 1 and to register, therein, reference images obtained by capturing this logo mark at various angles and distances. The in-use-state determining unit 16 may calculate the correlation value between an endoscopic image and each of the reference images obtained by capturing the logo mark and may determine whether the blood-vessel recognition probe 1 is in the in-use state on the basis of the magnitude relationship between the correlation value and a predetermined threshold. Since the image pattern of a living body differs from the logo mark, if the logo mark is included in the endoscopic image, a strong correlation value is obtained. The image library may be stored in the in-use-state determining unit 16 or may be stored in the storage unit 13.

### Second Embodiment

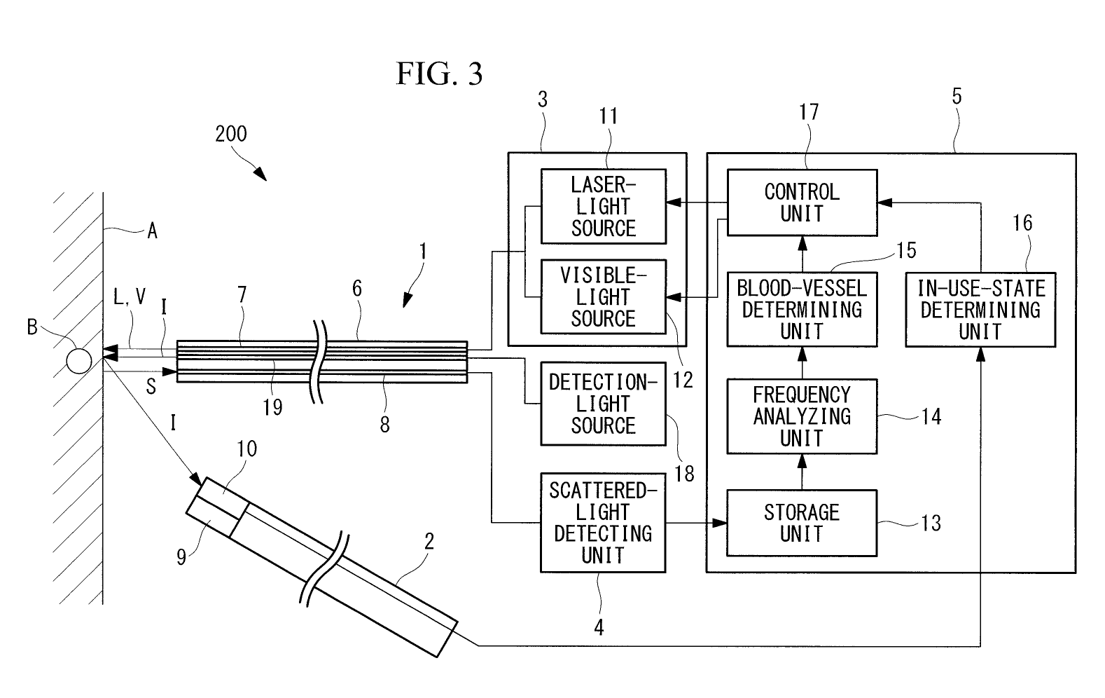

Next, a blood-vessel recognition system 200 according to a second embodiment of the present invention will be described with reference to FIG. 3.

In this embodiment, differences from the first embodiment will be described, identical reference signs are assigned to configurations common to those in the first embodiment, and a description thereof will be omitted.

As shown in FIG. 3, the blood-vessel recognition system 200 of this embodiment differs from the first embodiment in that the image acquisition unit (detection-light detecting unit) 10 of the endoscope 2 captures detection light I radiated onto the living tissue A from the blood-vessel recognition probe 1, and the in-use-state determining unit 16 determines whether the blood-vessel recognition probe 1 is in the in-use state on the basis of the presence or absence of an image of the detection light I in an endoscopic image.

Specifically, the blood-vessel recognition system 200 is further provided with a detection-light source 18 that outputs the detection light I. The blood-vessel recognition probe 1 is further provided with a detection optical fiber (detection-light emitting unit) 19.

The detection light I is light that has a lower intensity than the intensity of the laser light L, which is output from the laser-light source 11 to recognize the blood vessel B. The detection-light source 18 outputs the pulsed detection light I at predetermined time intervals. The detection-light source 18 constantly outputs the detection light I, independently of control of the other light sources 11 and 12 performed by the control unit 17.

A distal end of the detection optical fiber 19 is disposed in the vicinity of the distal end of the probe body 6, and a base end of the detection optical fiber 19 is connected to the detection-light source 18. The detection light I output from the detection-light source 18 is intermittently emitted from the distal end of the detection optical fiber 19. It is preferred that the detection light I be radiated onto the living tissue A at substantially the same position as the laser light L. Therefore, the distal end of the detection optical fiber 19 is disposed in the vicinity of the distal end of the irradiation optical fiber 7, so that the detection light I is emitted substantially parallel to the laser light L.

The image acquisition unit 10 of the endoscope 2 has sensitivity at least with respect to the detection light I and acquires an image of the detection light I emitted from the detection optical fiber 19 toward the living tissue A. In this embodiment, only an image of the detection light I may be acquired by the image acquisition unit 10, without radiating illumination light from the illumination unit 9 onto the living tissue A. An endoscopic image acquired by the image acquisition unit 10 is sent to the in-use-state determining unit 16.

When an image of the detection light I is included in the endoscopic image, the luminance value of the endoscopic image temporally changes regularly at time intervals equivalent to the time intervals at which the detection light I is output. In contrast to this, when an image of the detection light I is not included in the endoscopic image, the luminance value of the endoscopic image becomes substantially constant or temporally changes irregularly. The in-use-state determining unit 16 can determine whether an image of detection light I is included in an endoscopic image on the basis of the temporal change in the luminance value of the endoscopic image received from the image acquisition unit 10.

Specifically, the in-use-state determining unit 16 calculates the luminance value (for example, the average luminance value) of the endoscopic image and stores the calculated luminance value in time series, thereby obtaining luminance-value time-series data. When the luminance value is increased, in the luminance-value time-series data, at time intervals equivalent to the time intervals of the detection light I, the in-use-state determining unit 16 determines that the blood-vessel recognition probe 1 is in the in-use state and sends a TRUE signal to the control unit 17. On the other hand, when an increase in the luminance value at the above-described time intervals does not exist in the luminance-value time-series data, the in-use-state determining unit 16 determines that the blood-vessel recognition probe 1 is not in the in-use state and sends a FALSE signal to the control unit 17.

Next, the operation of the thus-configured blood-vessel recognition system 200 will be described.

According to the blood-vessel recognition system 200 of this embodiment, the detection light I is intermittently emitted, the in-use-state determining unit 16 determines whether the blood-vessel recognition probe 1 is in the in-use state according to whether an image of the detection light I is included in an endoscopic image, and, only when an image of the detection light I is included in an endoscopic image, the control unit 17 causes laser light L to be emitted.

Before the blood-vessel recognition probe 1 is inserted into the body, because the detection light I is not observed by the endoscope 2, emission of the laser light L is stopped. When the blood-vessel recognition probe 1 is inserted into the body, an image of the detection light I appears in an endoscopic image, and emission of the laser light L is started. Then, when the recognition operation of the blood vessel B using the blood-vessel recognition probe 1 is finished, and the blood-vessel recognition probe 1 is removed from the inside of the body, the image of the detection light I disappears from the endoscopic image, and the emission of the laser light L is stopped.

In this way, only when the blood-vessel recognition probe 1 is in the in-use state, in which laser light L is radiated onto the living tissue A to perform recognition of the blood vessel B in the body, the detection light I is observed by the endoscope 2. Therefore, whether the blood-vessel recognition probe 1 is in the in-use state can be accurately determined on the basis of an endoscopic image. Accordingly, there is an advantage in that, when the laser light L is not needed, e.g., when the blood-vessel recognition probe 1 is disposed outside the body, emission of the laser light L is automatically stopped, thus making it possible to automatically prevent the strong laser light L from being unnecessarily emitted.

When the blood-vessel recognition probe 1 is not in the in-use state, instead of stopping emission of the laser light L, the control unit 17 may reduce the intensity of the laser light L and to continue to cause the weak laser light L to be emitted.

In this embodiment, the image acquisition unit 10 may acquire images of both the blood-vessel recognition probe 1 and the detection light I, and the in-use-state determining unit 16 may determine whether the blood-vessel recognition probe 1 is in the in-use state on the basis of whether images of both the blood-vessel recognition probe 1 and the detection light I are included in an endoscopic image.

For example, when another treatment tool that has a shape similar to that of the blood-vessel recognition probe 1 is inserted into the body, instead of the blood-vessel recognition probe 1, there is a possibility that this other treatment tool is incorrectly determined to be the blood-vessel recognition probe 1 from the endoscopic image. In such a case, whether the blood-vessel recognition probe 1 is in the in-use state is more accurately determined on the basis of the presence or absence of an image of the detection light I, thus making it possible to appropriately control emission of the laser light L.

In this embodiment, although the detection-light source 18 intermittently outputs the detection light I, the detection light I merely needs to have a property different from that of the laser light L such that the detection light I can be detected in a clearly distinct fashion from the laser light L.

The property of the detection light I is, for example, a temporal change in the intensity, an intensity distribution in the cross section intersecting the optical axis, or a wavelength.

Specifically, with respect to the laser light L, which is output at a constant intensity during the in-use state, the detection light I may be intermittently output, as described above, or the intensity of the detection light I may be temporally changed in a predetermined pattern.

Alternatively, the detection-light source 18 may output detection light I having an intensity distribution in a predetermined pattern in a transverse cross section intersecting the optical axis. An image of the predetermined pattern is formed in an area, on the living tissue A, irradiated with the detection light I. Therefore, an image of the detection light I is acquired by the endoscope 2, and the presence or absence of an image of the detection light I having the predetermined pattern in an endoscopic image is determined through image recognition, thereby making it possible to determine whether the blood-vessel recognition probe 1 is in the in-use state.

Alternatively, the detection-light source 18 may be configured to output detection light I having a particular wavelength different from the wavelength of the laser light L, and the image acquisition unit 10 may be configured to acquire an image of only light having the wavelength of the detection light I.

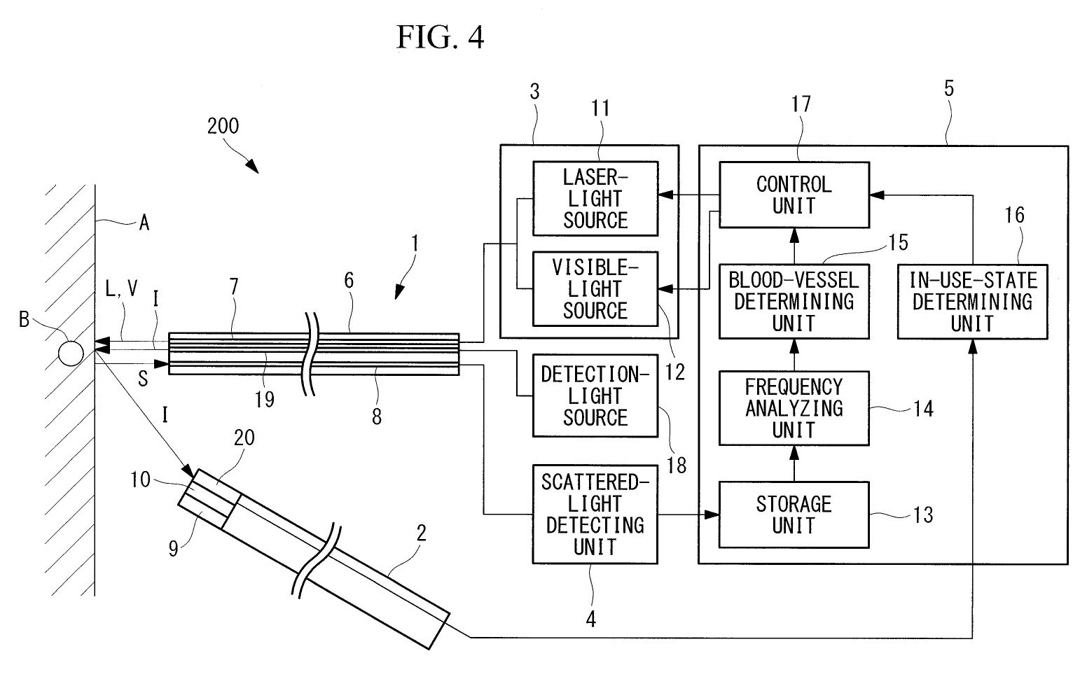

In this embodiment, although the detection light I is detected by using the image acquisition unit 10, instead of this, as shown in FIG. 4, it is also possible to provide, on the endoscope 2, a detection-light detecting unit 20 that is dedicated to detect the detection light I.

The detection-light detecting unit 20 is provided with, for example, a photodetector provided at the distal end of the endoscope 2, so as to detect detection light I reflected at the living tissue A. A wavelength filter that transmits only light having the wavelength of the detection light I may be provided in the previous stage of the photodetector, so that the detection light I can be accurately detected.

By doing so, the in-use-state determining unit 16 can determine whether the blood-vessel recognition probe 1 is in the in-use state on the basis of the intensity of the detection light I detected by the detection-light detecting unit 20.

In this embodiment, although the detection-light detecting unit 10, 20 is provided on the endoscope 2, instead of this, the detection-light detecting unit 10, 20 may be provided at a distal end portion of a desired device.

The desired device may be a dedicated device for detecting the detection light I or may be a treatment device that is used while being inserted into the body together with the blood-vessel recognition probe 1.

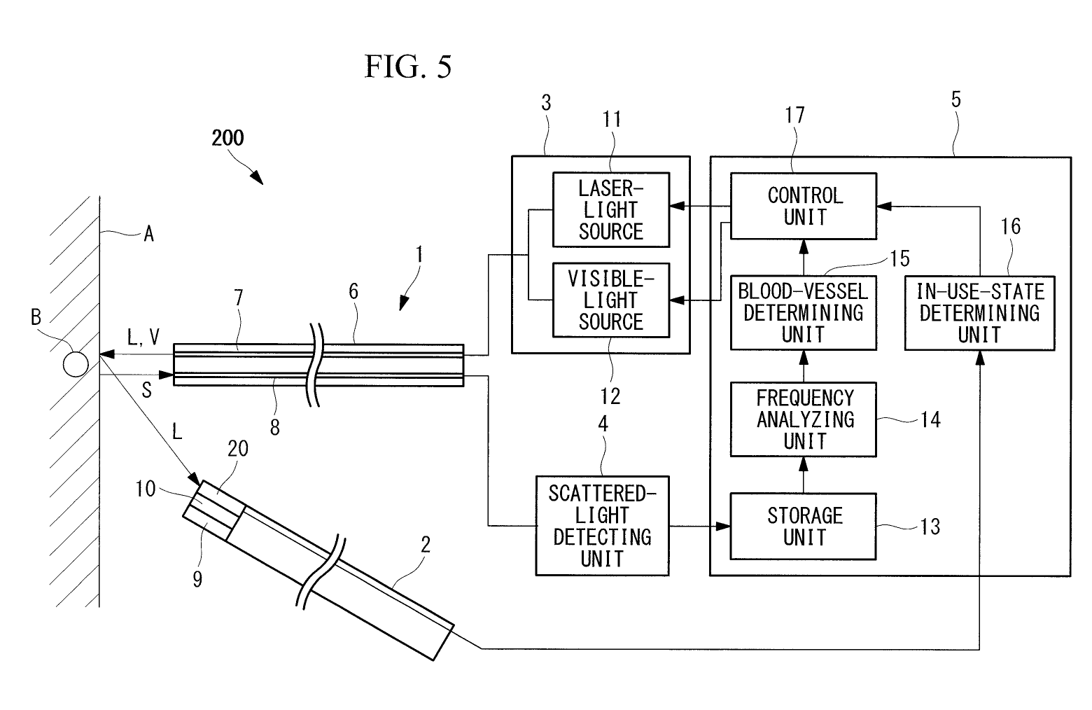

In this embodiment, although the detection-light source 18, which is separate from the laser-light source 11, is provided, instead of this, as shown in FIG. 5, the laser-light source 11 may also serve as the detection-light source 18.

In this case, the laser-light source 11 can change the intensity of the laser light L between a first intensity and a second intensity that is smaller than the first intensity. The first intensity is the intensity of the laser light L to be used in the recognition operation of the blood vessel B.

When the blood-vessel recognition probe 1 is in the in-use state, the laser light L reflected at the living tissue A is detected by the detection-light detecting unit 20. When the laser light L is detected by the detection-light detecting unit 20, the in-use-state determining unit 16 determines that the blood-vessel recognition probe 1 is in the in-use state, and the control unit 17 sets, at the first intensity, the intensity of the laser light L to be output from the laser-light source 11. On the other hand, when the laser light L is not detected by the detection-light detecting unit 20, the in-use-state determining unit 16 determines that the blood-vessel recognition probe 1 is not in the in-use state, and the control unit 17 sets, at the second intensity, the intensity of the laser light L to be output from the laser-light source 11.

By doing so, when the blood-vessel recognition probe 1 is not in the in-use state, the intensity of the laser light L can be weakened.

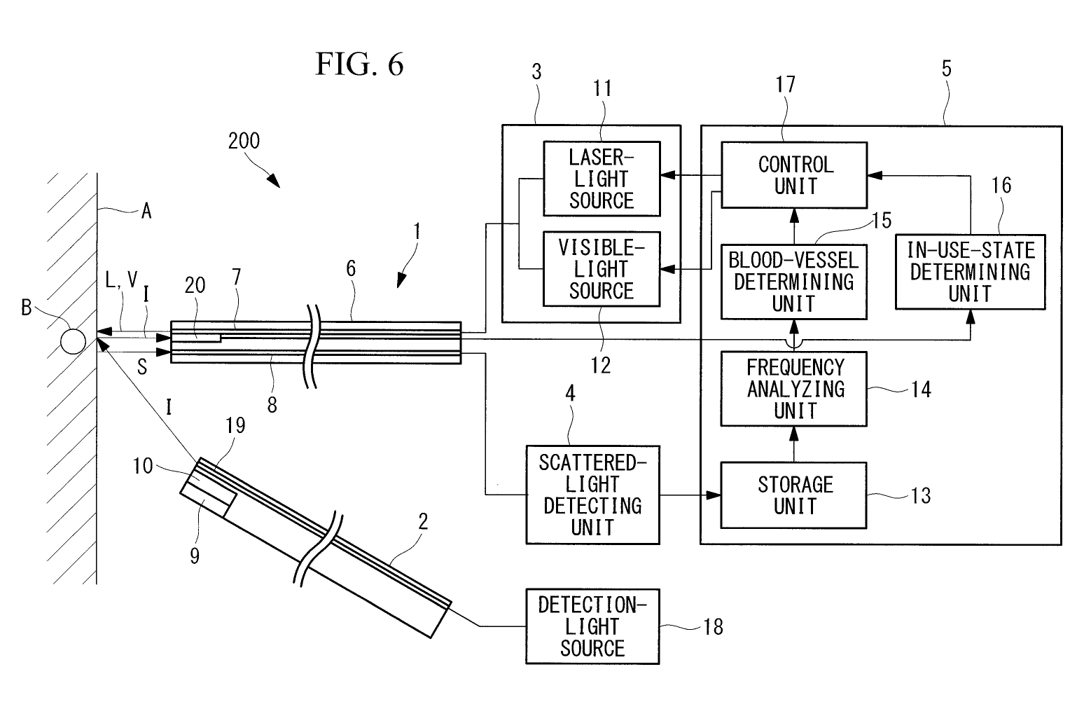

In this embodiment, although the detection optical fiber 19 is provided on the blood-vessel recognition probe 1, and the detection-light detecting unit 10, 20 is provided on the endoscope 2, instead of this, as shown in FIG. 6, the detection optical fiber 19 may be provided on the endoscope 2, and the detection-light detecting unit 20 may be provided on the blood-vessel recognition probe 1.

By doing so, only when the blood-vessel recognition probe 1 is in the in-use state, the detection light I emitted from the detection optical fiber 19 of the endoscope 2 can be detected by the detection-light detecting unit 20 of the blood-vessel recognition probe 1.

### Third Embodiment

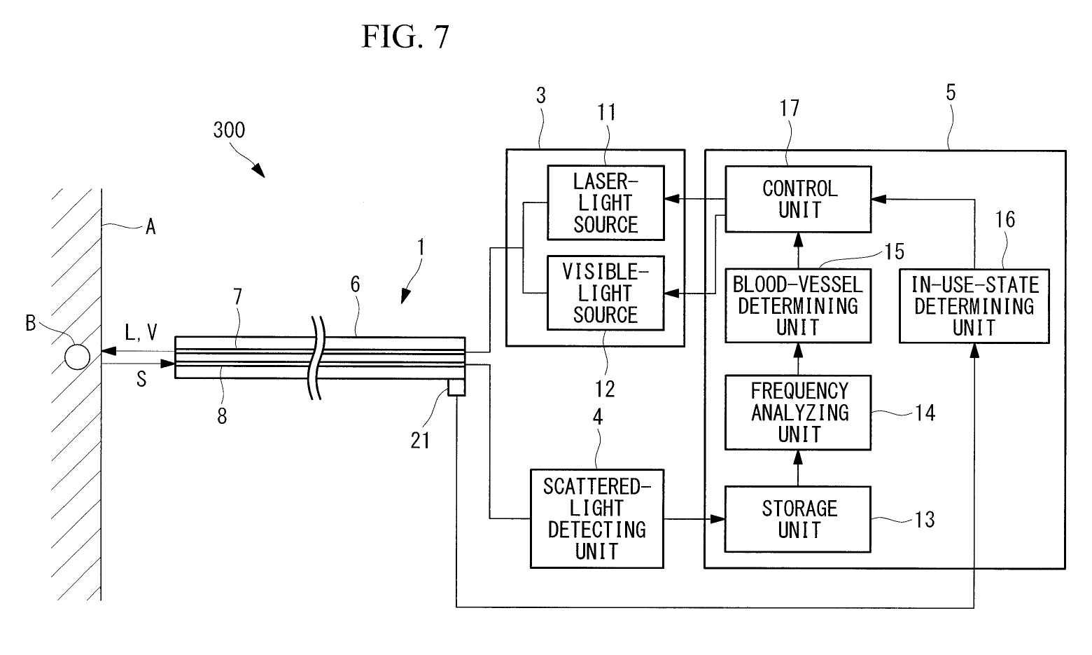

Next, a blood-vessel recognition system 300 according to a third embodiment of the present invention will be described with reference to FIG. 7.

In this embodiment, differences from the first embodiment will be described, identical reference signs are assigned to configurations common to those in the first embodiment, and a description thereof will be omitted.

As shown in FIG. 7, the blood-vessel recognition system 300 of this embodiment differs from the first embodiment in that an orientation detecting unit 21 that is provided on the probe body 6 is provided instead of the endoscope 2, and the in-use-state determining unit 16 determines whether the blood-vessel recognition probe 1 is in the in-use state on the basis of the orientation of the probe body 6 detected by the orientation detecting unit 21.

The orientation detecting unit 21 is a gravity sensor, for example, and detects, as the orientation of the probe body 6, an angle of the probe body 6 in the longitudinal direction with respect to the direction of gravity. The orientation detecting unit 21 sends information of the detected orientation to the in-use-state determining unit 16.

The in-use-state determining unit 16 determines whether the probe body 6 is in the in-use state on the basis of the information of the orientation received from the orientation detecting unit 21. In the in-use state, the probe body 6 is inserted, from an upper side, into the body of a patient lying in the bed. Therefore, the probe body 6 in the in-use state is disposed parallel to or inclined with respect to the direction of gravity, with the distal end thereof being directed downward. When the probe body 6 is disposed in an orientation in which the distal end thereof is directed downward, and the longitudinal direction of the probe body 6 is parallel to or inclined with respect to the direction of gravity, the in-use-state determining unit 16 determines that the probe body 6 is in the in-use state and sends a TRUE signal to the control unit 17. On the other hand, when the probe body 6 is disposed in an orientation in which the distal end thereof is directed upward or in the horizontal direction, the in-use-state determining unit 16 determines that the probe body 6 is not in the in-use state and sends a FALSE signal to the control unit 17.

Next, the operation of the thus-configured blood-vessel recognition system 300 will be described.

According to the blood-vessel recognition system 300 of this embodiment, the orientation detecting unit 21 detects the orientation of the probe body 6, the in-use-state determining unit 16 determines whether the blood-vessel recognition probe 1 is in the in-use state on the basis of the orientation of the probe body 6, and, only when the probe body 6 is disposed with the distal end thereof being directed downward, the control unit 17 causes laser light L to be emitted.

Therefore, when the surgeon grasps the probe body 6 with the distal end of the probe body 6 being directed downward, in order to insert the probe body 6 into the body, emission of laser light L is started, and the laser light L is continuously emitted while the probe body 6 is inserted into the body. When the surgeon removes the probe body 6 from the inside of the body after use and disposes the probe body 6 in a substantially horizontal direction or directs the distal end thereof upward, emission of the laser light L is stopped.

In this way, in the in-use state, in which recognition of the blood vessel B is performed, the blood-vessel recognition probe 1 is disposed in a particular orientation. Therefore, it is possible to determine whether the blood-vessel recognition probe 1 is in the in-use state on the basis of the orientation of the blood-vessel recognition probe 1. Accordingly, there is an advantage in that, when the laser light L is not needed, emission of the laser light L is automatically stopped, thus making it possible to automatically prevent the strong laser light L from being unnecessarily emitted.

When the blood-vessel recognition probe 1 is not in the in-use state, instead of stopping emission of the laser light L, the control unit 17 may reduce the intensity of the laser light L and continue to cause the weak laser light L to be emitted.

The orientation detecting unit 21 may be used in combination with the endoscope 2, which is described in the first and second embodiments. In this case, the in-use-state determining unit 16 determines whether the blood-vessel recognition probe 1 is in the in-use state on the basis of both the endoscopic image and the orientation of the blood-vessel recognition probe 1.

By doing so, it is possible to more accurately determine whether the blood-vessel recognition probe 1 is in the in-use state.

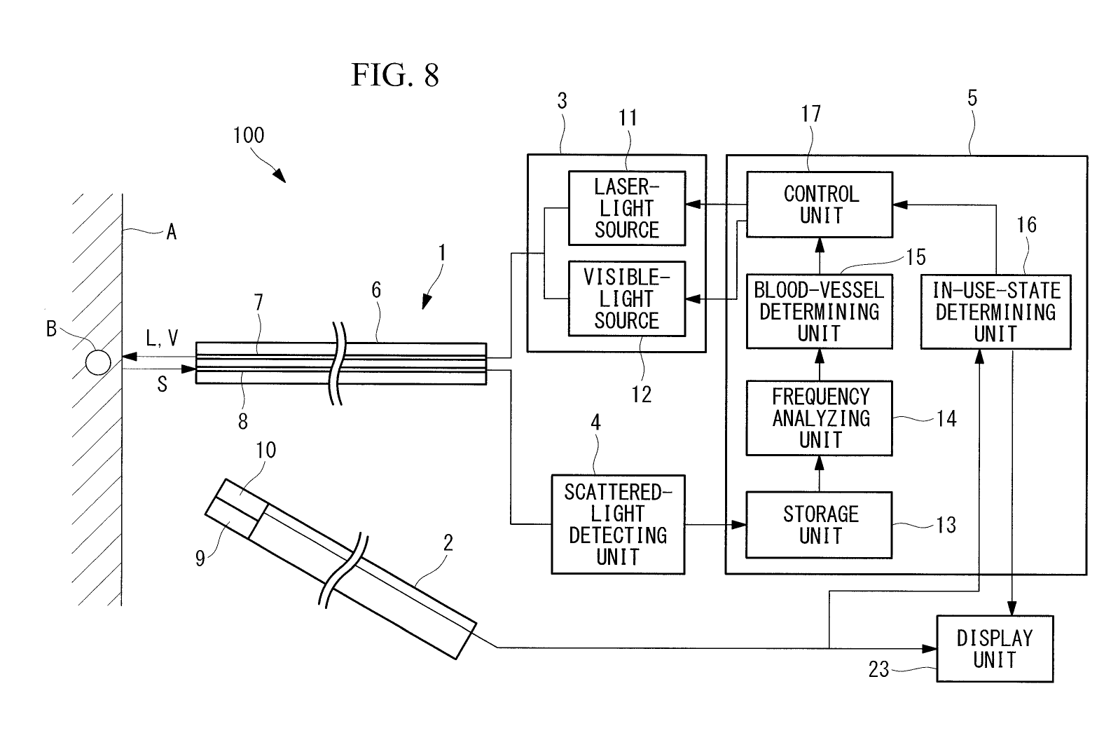

In the first to third embodiments, as shown in FIG. 8, it is also possible to further provide a display unit 23 that displays an in-use-state indication that indicates a determination result obtained by the in-use-state determining unit 16. FIG. 8 shows, as an example, the blood-vessel recognition system 100 of the first embodiment, which includes the display unit 23. By doing so, whether the blood-vessel recognition probe 1 is in the in-use state, i.e., whether the laser light L is being emitted, can be clearly recognized by the user. The in-use-state indication may be displayed on the display unit 23 together with an endoscopic image acquired by the endoscope 2.

As a result, the following aspect is read by the above described embodiment of the present invention.

An aspect of the present invention provides a blood-vessel recognition system including: a laser-light source; a blood-vessel recognition probe that has a probe body that is inserted into a body and a laser-light emitting unit that is provided on the probe body and that emits laser light supplied from the laser-light source; an in-use-state determining unit that determines whether the blood-vessel recognition probe is in an in-use state in which the laser light emitted from the laser-light emitting unit is radiated onto living tissue in the body; and a control unit that controls the laser-light source on the basis of a determination result obtained by the in-use-state determining unit, such that the intensity of the laser light is reduced when the blood-vessel recognition probe is not in the in-use state, compared with when the blood-vessel recognition probe is in the in-use state.

According to the present aspect, when the probe body is inserted into the body, and the laser light emitted from the laser-light emitting unit is radiated onto the living tissue, the laser light is scattered by the living tissue, thus producing scattered light. The frequency of the scattered light scattered by blood flowing in a blood vessel in the living tissue is shifted with respect to the frequency of the laser light due to the Doppler shift. Therefore, it is possible to recognize a blood vessel in the living tissue on the basis of the frequency of scattered light received by a light-receiving part.

In this case, the in-use-state determining unit determines whether the blood-vessel recognition probe is in the in-use state, in which the laser light is radiated onto the living tissue to perform a blood-vessel recognition operation. If the blood-vessel recognition probe is not in the in-use state, the control unit reduces the intensity of the laser light output from the laser-light source. Accordingly, it is possible to automatically prevent the strong laser light from being unnecessarily emitted.

The above-described aspect may further include an endoscope that is inserted into the body, wherein the in-use-state determining unit may determine that the blood-vessel recognition probe is in the in-use state when an image of the blood-vessel recognition probe is included in an endoscopic image acquired by the endoscope.

When the recognition operation of a blood vessel in a treatment area in the body is performed by means of the blood-vessel recognition probe while observing the treatment area with the endoscope, if the blood-vessel recognition probe is in the in-use state, an image of a distal end portion of the blood-vessel recognition probe disposed in the vicinity of the treatment area is included in an endoscopic image. Therefore, whether the blood-vessel recognition probe is in the in-use state can be determined on the basis of the presence or absence of an image of the blood-vessel recognition probe in the endoscopic image.

The above-described aspect may further include: a detection-light source that outputs detection light having a lower intensity than the intensity of the laser light, which is output from the laser-light source when the blood-vessel recognition probe is in the in-use state; and a detection-light detecting unit that detects the detection light radiated onto the living tissue, wherein the blood-vessel recognition probe may be provided with a detection-light emitting unit that is provided on the probe body and that radiates, onto the living tissue, the detection light supplied from the detection-light source; and the in-use-state determining unit may determine that the blood-vessel recognition probe is in the in-use state when the detection-light detecting unit detects the detection light.

When the blood-vessel recognition probe is in the in-use state, detection light emitted from the detection-light emitting unit is radiated onto the living tissue. Therefore, whether the blood-vessel recognition probe is in the in-use state can be determined depending on whether the detection light is radiated onto the living tissue.

The above-described aspect may further include: an endoscope that is inserted into the body; a detection-light source that outputs detection light having a lower intensity than the intensity of the laser light, which is output from the laser-light source when the blood-vessel recognition probe is in the in-use state; and a detection-light detecting unit that is provided on the probe body and that detects the detection light radiated onto the living tissue, wherein the endoscope may be provided with a detection-light emitting unit that radiates, onto the living tissue, the detection light supplied from the detection-light source; and the in-use-state determining unit may determine that the blood-vessel recognition probe is in the in-use state when the detection-light detecting unit detects the detection light.

When the recognition operation of a blood vessel in a treatment area in the body is performed by means of the blood-vessel recognition probe while observing the treatment area with the endoscope, if the blood-vessel recognition probe is in the in-use state, detection light emitted from the detection-light emitting unit of the endoscope is detected by the detection-light detecting unit. Accordingly, whether the blood-vessel recognition probe is in the in-use state can be determined.

In the above-described aspect, the detection-light source may output the detection light, which has a property different from that of the laser light.

By doing so, the detection light can be accurately detected in a distinct fashion from the laser light.

In the above-described aspect, the detection-light source may output the detection light whose intensity temporally changes; and the in-use-state determining unit may determine whether the blood-vessel recognition probe is in the in-use state on the basis of the temporal change in the intensity of the detection light detected by the detection-light detecting unit.

By doing so, the detection light radiated onto the living tissue can be accurately detected.

In the above-described aspect, the detection-light source may output the detection light, which has an intensity distribution having a predetermined pattern in the cross section intersecting the optical axis; the detection-light detecting unit may acquire an image of the living tissue; and the in-use-state determining unit may determine that the blood-vessel recognition probe is in the in-use state when an image of the predetermined pattern of the detection light is included in the image acquired by the detection-light detecting unit.

By doing so, the detection light radiated onto the living tissue can be accurately detected.

In the above-described aspect, the laser-light source may be capable of changing the intensity of the laser light between a first intensity and a second intensity that is lower than the first intensity; and the detection-light source may be formed of the laser-light source.

By doing so, the laser light having the second intensity is used as detection light, thereby making it possible to eliminate addition of a light source.

The above-described aspect may further include an orientation detecting unit that is provided on the probe body and that detects an orientation of the probe body, wherein the in-use-state determining unit may determine whether the blood-vessel recognition probe is in the in-use state on the basis of the orientation of the probe body detected by the orientation detecting unit.

In the in-use state, the blood-vessel recognition probe is disposed in a particular orientation. Therefore, whether the blood-vessel recognition probe is in the in-use state can be determined on the basis of the orientation of the probe body.

The above-described aspect may further include a display unit that displays an in-use-state indication indicating a determination result obtained by the in-use-state determining unit.

By doing so, it is possible for a surgeon to clearly recognize whether strong laser light is emitted.

### REFERENCE SIGNS LIST

1 blood-vessel recognition probe
2 endoscope
3 light source unit
4 scattered-light detecting unit
5 control device
6 probe body
7 irradiation optical fiber (laser-light emitting unit)
8 light-receiving optical fiber
9 illumination unit
10 image acquisition unit (detection-light detecting unit)
11 laser-light source
12 visible-light source
13 storage unit
14 frequency analyzing unit
15 blood-vessel determining unit
16 in-use-state determining unit
17 control unit
18 detection-light source
19 detection optical fiber (detection-light emitting unit)
20 detection-light detecting unit
21 orientation detecting unit
23 display unit
100, 200, 300 blood-vessel recognition system
L laser light
S scattered light
V visible light
I detection light

1. A blood-vessel recognition system comprising:
a laser-light source;
a blood-vessel recognition probe that has a probe body that is inserted into a body and a laser-light emitting unit that is provided on the probe body and that emits laser light supplied from the laser-light source;
an in-use-state determining unit that determines whether the blood-vessel recognition probe is in an in-use state in which the laser light emitted from the laser-light emitting unit is radiated onto living tissue in the body; and
a control unit that controls the laser-light source on the basis of a determination result obtained by the in-use-state determining unit, such that the intensity of the laser light is reduced when the blood-vessel recognition probe is not in the in-use state, compared with when the blood-vessel recognition probe is in the in-use state.

2. A blood-vessel recognition system according to claim 1, further comprising an endoscope that is inserted into the body,
wherein the in-use-state determining unit determines that the blood-vessel recognition probe is in the in-use state when an image of the blood-vessel recognition probe is included in an endoscopic image acquired by the endoscope.

3. A blood-vessel recognition system according to claim 1, further comprising:
a detection-light source that outputs detection light having a lower intensity than the intensity of the laser light, which is output from the laser-light source when the blood-vessel recognition probe is in the in-use state; and
a detection-light detecting unit that detects the detection light radiated onto the living tissue,
wherein the blood-vessel recognition probe is provided with a detection-light emitting unit that is provided on the probe body and that radiates, onto the living tissue, the detection light supplied from the detection-light source; and
the in-use-state determining unit determines that the blood-vessel recognition probe is in the in-use state when the detection-light detecting unit detects the detection light.

4. A blood-vessel recognition system according to claim 1, further comprising:
an endoscope that is inserted into the body;
a detection-light source that outputs detection light having a lower intensity than the intensity of the laser light, which is output from the laser-light source when the blood-vessel recognition probe is in the in-use state; and
a detection-light detecting unit that is provided on the probe body and that detects the detection light radiated onto the living tissue,
wherein the endoscope is provided with a detection-light emitting unit that radiates, onto the living tissue, the detection light supplied from the detection-light source; and
the in-use-state determining unit determines that the blood-vessel recognition probe is in the in-use state when the detection-light detecting unit detects the detection light.

5. A blood-vessel recognition system according to claim 3, wherein the detection-light source outputs the detection light, which has a property different from that of the laser light.

6. A blood-vessel recognition system according to claim 5,
wherein the detection-light source outputs the detection light whose intensity temporally changes; and
the in-use-state determining unit determines whether the blood-vessel recognition probe is in the in-use state on the basis of the temporal change in the intensity of the detection light detected by the detection-light detecting unit.

7. A blood-vessel recognition system according to claim 3,
wherein the detection-light source outputs the detection light, which has an intensity distribution having a predetermined pattern in the cross section intersecting the optical axis;
the detection-light detecting unit acquires an image of the living tissue; and
the in-use-state determining unit determines that the blood-vessel recognition probe is in the in-use state when an image of the predetermined pattern of the detection light is included in the image acquired by the detection-light detecting unit.

8. A blood-vessel recognition system according to claim 3,
wherein the laser-light source can change the intensity of the laser light between a first intensity and a second intensity that is lower than the first intensity; and
the detection-light source is formed of the laser-light source.

9. A blood-vessel recognition system according to claim 1, further comprising an orientation detecting unit that is provided on the probe body and that detects an orientation of the probe body,
wherein the in-use-state determining unit determines whether the blood-vessel recognition probe is in the in-use state on the basis of the orientation of the probe body detected by the orientation detecting unit.

10. A blood-vessel recognition system according to claim 1, further comprising a display unit that displays an in-use-state indication indicating a determination result obtained by the in-use-state determining unit.
